SEQUENCE LISTING (8 sequences)

SEQ ID NO: 1 - DNA, 27 bp, synthetic construct
  source 1..27: mol_type = other DNA
acctgggggagtattgcggaggaaggt

SEQ ID NO: 2 - DNA, 40 bp, synthetic construct
  source 1..40: mol_type = other DNA
acgaccgtgtgtgttgctctgtaacagtgtccattgtcgt

SEQ ID NO: 3 - DNA, 34 bp, synthetic construct
  source 1..34: mol_type = other DNA
accgtgtgtgttgctctgtaacagtgtccattgt

SEQ ID NO: 4 - DNA, 28 bp, synthetic construct
  source 1..28: mol_type = other DNA
gtgtgtgttgctctgtaacagtgtccat

SEQ ID NO: 5 - DNA, 27 bp, synthetic construct
  source 1..27: mol_type = other DNA
attattttttatttatttttattttat

SEQ ID NO: 6 - DNA, 40 bp, synthetic construct
  source 1..40: mol_type = other DNA
attattttttatttatttttattttattttattttttatt

SEQ ID NO: 7 - DNA, 45 bp, synthetic construct
  source 1..45: mol_type = other DNA
cgagggtaccgcaatagtacttattgttcgcctattgtgggtcgg

SEQ ID NO: 8 - DNA, 39 bp, synthetic construct
  source 1..39: mol_type = other DNA
attattttttatttatttttattttattttattttttat

(19) United States
(12) Patent Application Publication
(10) Pub. No.: US 2026/0098860 A1
(43) Pub. Date: Apr. 9, 2026

(54) SENSOR, KIT, AND METHOD FOR BIOMARKER DETECTION IN SALIVA

(71) Applicant: SOCIETE DE COMMERCIALISATION DES PRODUITS DE LA RECHERCHE APPLIQUEE SOCPRA SCIENCES ET GENIE, S.E.C., Sherbrooke (CA)

(72) Inventors: Philippe Dauphin DUCHARME, Sherbrooke (CA); Minh-dat NGUYEN, Sherbrooke (CA); Indrani BANERJEE, Basking Ridge, NJ (US); Donghui WU, Bridgewater, NJ (US); Laurence DU-THUMM, Princeton, NJ (US)

(21) Appl. No.: 19/111,827

(22) PCT Filed: Sep. 13, 2023
(86) PCT No.: PCT/CA2023/051213

§ 371 Date: Mar. 14, 2025

Related U.S. Application Data
(60) Provisional application No. 63/375,802, filed on Sep. 15, 2022.

(51) Int. Cl. G01N 33/53; G01N 27/327; G01N 33/543; G01N 33/66; G01N 33/68

(52) U.S. Cl. CPC G01N 33/5308; G01N 27/3276; G01N 27/3277; G01N 33/5438; G01N 33/66; G01N 33/68

(57) ABSTRACT

It is provided a sensor for measuring a biomarker or metabolite level such as glucose level in a fluid sample such as saliva comprising a sensing element comprising a redox reporter attached to at least one aptamer that binds to the biomarker or metabolite, and a solid surface where the sensing element is attached thereto, wherein upon binding of the biomarker or metabolite onto to the aptamer, the redox reporter undergoes a binding-induced change in electron transfer. Measuring the biomarker or metabolite level in the saliva of a patient will allow health monitoring in the patient such as increased risk of gum disease or diabetes in the patient.

### CROSS-REFERENCE TO RELATED APPLICATION

The present application is claiming priority from U.S. Provisional Application No. 63/375,802 filed Sep. 15, 2022, the content of which is hereby incorporated by reference in its entirety.

### TECHNICAL FIELD

It is provided a sensor, kit, and method for measuring a biomarker level in saliva.

### BACKGROUND

Various medical conditions and monitoring of health status need regular or frequent patient-operated testing to monitor conditions or well-being of patients.

Sensors based on electrochemical processes can be used to detect a biological substance by using a transducing element to convert a detection event into a signal for processing and/or display. Biosensors can use biological materials as the biologically sensitive component, e.g., enzymes, antibodies, or nucleic acids. Biosensors typically use a transducer element to transform a signal resulting from the detection of an analyte by the biologically sensitive component into a different signal that can be addressed by optical, electronic or other means.

One example of a target analyte is the measurement of glucose level of diabetes patients. Diabetes (diabetes mellitus) is a metabolic disease associated with high blood sugar due to insufficient production of insulin by the body or inadequate response by cells to the insulin that is produced. Good blood glucose control is also known to be important to prevent gingivitis from developing. Diabetic people have a higher risk of developing gum disease.

Accordingly, there is still a need to be provided with new means to measure biomarker levels in bodily fluids.

### SUMMARY

It is provided a sensor for measuring a target molecule in a saliva sample comprising a sensing element comprising a redox reporter attached to at least one aptamer that binds to a salivary metabolite; and a solid surface where the sensing element is attached thereto, wherein upon binding of the salivary metabolite onto to the aptamer, the redox reporter undergoes a binding-induced change in electron transfer.

It is particularly provided a sensor for measuring a target molecule level in a fluid sample comprising a sensing element comprising a redox reporter attached to at least one aptamer that binds to glucose, and a solid surface where the sensing element is attached thereto, wherein upon binding of glucose onto to the aptamer, the redox reporter undergoes a binding-induced change in electron transfer.

In an embodiment, the aptamer is attached to the solid surface via a thiol molecule.

In another embodiment, the redox reporter is a methylene blue molecule.

In a further embodiment, the at least one aptamer comprises a nucleotide sequence depicted as SEQ ID NO: 3 or SEQ ID NO: 4, or a combination thereof.

In a further embodiment, the at least one aptamer comprises SEQ ID NO: 7.

In an additional embodiment, the sensor encompassed herewith further comprises at least one aptamer binding to a different target molecule.

In an embodiment, the target molecule is glucose.

In another embodiment, the target is vancomycin.

In a further embodiment, the target is adenosine triphosphate (ATP), carnitine, lactate, lactic acid, malate, malic acid, maltose, or adenosine monophosphate (AMP).

In an embodiment, the carnitine is 2-Methylbutyryl carnitine, Butyryl carnitine, Isobutyryl carnitine, or Propionyl carnitine.

In an embodiment, the at least one aptamer binding to a different target molecule comprises the nucleotide sequence depicted as SEQ ID NO: 1.

In a further embodiment, the solid surface is an electrode.

In another embodiment, the electrode is a gold electrode.

In a supplemental embodiment, the fluid sample is saliva.

In a further embodiment, the sensor element is used for an extended period.

In an embodiment, the sensor element is used for more than three days.

In another embodiment, the sensor element is regenerated and used for multiple measuring.

It is also provided a kit comprising the sensor element as defined herein and instruction for use.

In an embodiment, the kit encompassed herein further comprises a monitoring device.

In another embodiment, the monitoring device monitors the binding-induced change in electron transfer of the redox reporter.

In a further embodiment, the electron transfer is monitored via a change in the peak height of square-wave voltammograms.

In another embodiment, the monitoring device monitors the binding-induced change in electron transfer voltametrically or impedimetrically.

It is also provided a method of measuring at least one metabolite, such as glucose level, in a fluid sample from a patient comprising the steps of contacting the fluid sample with the sensor as defined herein, and monitoring the binding-induced change in electron transfer of the redox reporter indicating the presence of the at least one metabolite in the fluid sample.

In an embodiment, the method described herein further comprises the step of measuring the presence of at least one other analyte.

In an embodiment, measuring at least one metabolite level allows monitoring health in the patient.

In a further embodiment, measuring glucose level is indicative of the presence or progression of diabetes or increased risk of gum disease in the patient.

In an embodiment, the method encompassed is for measuring the at least one metabolite with the sensor for an extended period.

In an embodiment, the method encompassed is for measuring the at least one metabolite with the sensor for more than three days.

In another embodiment, the method encompassed herein further comprises the step of cleaning and storing the sensor and reusing the sensor for multiple measuring.

In a further embodiment, the metabolite is measured before and/or after the patient consumes food.

### BRIEF DESCRIPTION OF THE DRAWINGS

Reference will now be made to the accompanying drawings.

FIG. 1 illustrates in (A) that electrochemical aptamer-based biosensors are comprised of a methylene blue-modified aptamer sequence that is attached via thiol chemistry on a monolayer-coated gold electrode surface; and in (B) upon target recognition, the methylene blue redox reporter undergoes a binding-induced change in electron transfer which can, for example, easily be monitored via a change in the peak height of square-wave voltammograms.

FIG. 2 illustrates that the E-AB sensor signal response is dependent on square-wave frequency, as observed in (A) the ATP aptamer and parent glucose aptamer in (B) in working buffer and in undiluted human saliva over a broad range of square-wave frequencies. Error bars indicate the standard deviations observed across at least three independently fabricated and tested biosensors. pH of saliva sample is 7.25 in (A) and 7.74 in (B).

FIG. 3 illustrates E-AB sensor analytical performances (i.e., limit of detection and signal change) improve when deployed in undiluted saliva, in (A) sensor binding with the adenosine monophosphate-binding aptamer; in (B) sensors binding of glucose in saliva; and in (C) the improved glucose aptamer signal response with the three new variants.

FIG. 4 illustrates E-AB sensors binding adenosine monophosphate (A) and glucose (B) rapidly respond within 20 s when challenged with 100 nM of adenosine monophosphate and 5 pM of glucose. Error bars indicate the standard deviation of the mean measured across at least three independently fabricated sensors.

FIG. 5 illustrates that glucose-binding aptamer shows prolonged stability when interrogated continuously in undiluted saliva over a period of more than 3 days. Shading represents the standard deviation of the mean of 8 sensors continuously interrogated and independently fabricated.

FIG. 6 illustrates that E-AB biosensors can withstand potentially more than 10 cycles of rinse/target challenge steps directly in undiluted saliva. Shading represents the standard deviation of the mean of at least 4 sensors continuously interrogated and independently fabricated.

FIG. 7 illustrates that E-AB sensors show an enhanced performance when deployed in undiluted saliva with respect to when in buffer due to a unique combination of ionic species at given concentrations (top panels), as shown with the adenosine-monophosphate-binding E-AB sensors through an exposition to increasing amounts of the divalent cations, Ca²⁺ or Mg²⁺, and CD spectroscopy study confirmed mechanism that sensor signal enhancement in saliva was due to the presence of divalent ions, such as Ca²⁺ and Mg²⁺ (bottom panels).

FIG. 8 illustrates E-AB sensors' curve response being a square-wave frequency dependent, wherein shadings indicate the standard deviations observed across at least three independently fabricated and tested biosensors.

FIG. 9 illustrates that E-AB sensor display improved analytical performances when deployed in undiluted saliva. Error bars represent the standard deviation of the mean for at least three independently fabricated (too small to be shown in some cases).

FIG. 10A illustrates variations of glucose concentration as of function of time in saliva samples collected from the same individual at different moments during the same day measured colorimetrically using a standardized test or by using E-AB sensors. The glucose sensor responses after meal 4 h and 6 h are significantly different from the ones after lunch 30 min (*p=0.017 and ***p=5.42e-4, respectively, n=4 sensors where *p<0.05, **p<0.01, ***p<0.001, ns not significant) as determined via a paired samples T test. Shadings indicate the standard deviations observed across at least three independently fabricated and tested biosensors.

FIG. 10B illustrates variations of adenosine monophosphate concentrations as of function of time in saliva samples collected from the same individual at different moments during the same day using E-AB sensors. Shadings indicate the standard deviations observed across at least three independently fabricated and tested biosensors.

### DETAILED DESCRIPTION

In accordance with the present description, there is provided a sensor for measuring a biomarker, such as for example and not limited to glucose level, in a fluid sample, such as for example and not limited to saliva, comprising a sensing element comprising a redox reporter attached to at least one aptamer that binds to glucose; and a solid surface where the sensing element is attached thereto, wherein upon binding of glucose onto the aptamer, the redox reporter undergoes a binding-induced change in electron transfer.

Electrochemical aptamer-based biosensors (E-AB) have the potential to offer for point-of-need and convenient measurements of biomarkers directly in undiluted complex matrices. E-AB sensors encompassed herein are comprised of a redox reporter, e.g. but not limited to methylene blue, coupled to the aptamers. More specifically, it is proved a E-AB comprising a methylene blue-and-thiol-modified aptamer sequence. As encompassed herein, the aptamer can detect molecules or biomarkers, such as for example glucose, carnitines (e.g. 2-Methylbutyryl carnitine, Butyryl carnitine, Isobutyryl carnitine, Propionyl carnitine), lactate/lactic acid, malate/malic acid, Adenosine 5′-monophosphate (AMP), or maltose. Aptamers are short single stranded DNA (ssDNA) or RNA sequences that have been used as recognition receptors as alternative to antibodies in biosensing devices. Aptamers can be selected in vitro against a variety of targets including small molecules, metal ions and proteins using a process known as SELEX.

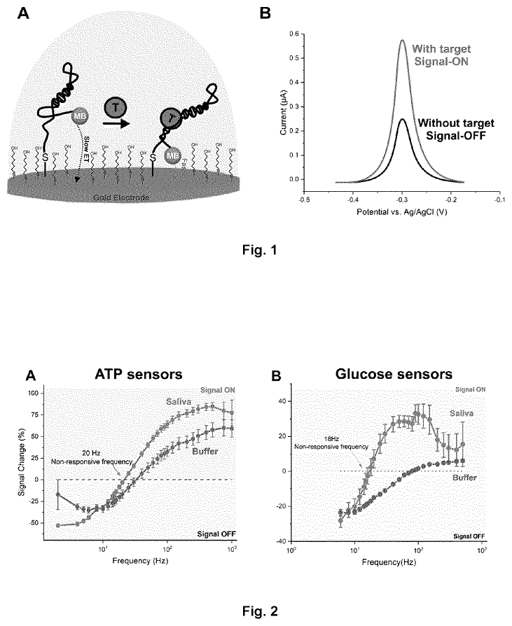

In addition, the E-AB also comprises a monolayer solid surface such as a coated gold electrode surface for aptamer attachment (see FIG. 1). It is encompassed that the electrode can be made from other known materials such as copper, graphite, titanium, brass, silver, and platinum, carbon, silicon.

As provided herein, the aptamers are attached to the solid surface via a thiol molecule. There is a vast library of thiols modified molecules that can be used to passivate gold electrode. There are different electrode treatments that can be done to gold electrodes to change its surface area and porosity.

Upon target binding, the attached redox reporter undergoes a binding-induced change in electron transfer which allows for a direct quantification of target concentrations (FIG. 1B).

As encompassed herein, a monitoring device can be coupled to the E-AB in order to monitor the binding-induced change in electron transfer of the redox reporter. This change can be monitored using various electrochemical techniques (i.e., voltammetrically or impedimetrically). As encompassed herein, the electron transfer is monitored via a change in the peak height of square-wave voltammograms.

Because E-AB sensors rely on an electrochemical signal transduction mechanism, they have shown deployment in undiluted whole blood of antibiotics, chemotherapeutics, drugs of abuse and proteins. Although blood is considered as a gold standard in most health exams, as a clinically informative biological fluid, saliva, in contrast, represents an attractive and non-invasive mean for diagnosis. To date, aside from SARS-COV-2, E-AB sensors have yet to demonstrate deployability in undiluted saliva.

Because E-AB sensors rely on an electrochemical signal transduction mechanism, they can be powered by miniaturized portable electronics.

Previously reported aptamers binding glucose and adenosine triphosphate (and also adenosine monophosphate) were tested in buffers in order to evaluate their abilities to readily deploy in undiluted unstimulated saliva. The ATP and AMP-binding aptamer is well known in the field and was further adapted in E-AB sensors. However, no demonstration was made of the applicability of this E-AB sensor in undiluted saliva. The “parent” glucose aptamer was previously published and incorporated in a field effect transistor platform which did not demonstrate measurements in undiluted complex matrices as it relies on a signal transduction scheme that is prone to electrode fouling. Through this, analytical figures of merit were determined (sensitivity, dynamic range, limit of quantification, limit of detection) of the biosensors in this complex matrix.

Aptamer sequences employed were purchased from Bio Basic Inc with HPLC purification. The sequences were not further purified and simply resuspended those at 100 UM in deionized water prior to aliquoting in 2 μL tubes. Bio Basic Inc chemically modified the 5′ end of aptamers with a thiol on a 6-carbon linker and the 3′ end with a carboxy-modified methylene blue attached to the DNA via the formation of an amide bond to a primary amine on a 6-carbon linker.

ATP aptamer:
(SEQ ID NO: 1)
5′—HS—SH—(CH₂)₆—ACC TGG GGG AGT ATT GCG
GAG GAA GGT—O—(CH₂)₆—NH—CO—(CH₂)₂—MB—3′
—Glucose parent aptamer (previously published
and modified with methylene blue and thiol):
(SEQ ID NO: 2)
5′—HS—SH—(CH₂)₆—ACG ACC GTG TGT GTT GCT CTG
TAA CAG TGT CCATTG TCG T—O—(CH₂)₆—NH—CO—
(CH₂)₂—MB—3′
Glucose 3—trunc aptamer
(new unreported aptamer):
(SEQ ID NO: 3)
5′—HS—SH—(CH₂)₆—ACC GTGTGT GTT GCT CTG TAA
CAG TGT CCA TTG T—O—(CH₂)₆—NH—CO—(CH₂)₂—MB—3′
Glucose 6—trunc aptamer (new unreported
aptamer):
(SEQ ID NO: 4)
5′—HS—SH—(CH₂)₆—GTG TGT GTT GCT CTG TAA CAG
TGT CCA T—O—(CH₂)₆—NH—CO—(CH₂)₂—MB—3′
T26 sequence (control sequence for ATP
aptamer):
(SEQ ID NO: 5)
5′—HS—SH—(CH₂)₆—ATT ATT TTT TAT TTA TTT
TTA TTT TAT—O—(CH₂)₆—NH—CO—(CH₂)₂—MB—3′
T40 sequence (control sequence for original
glucose aptamer):
(SEQ ID NO: 6)
5′—HS—SH—(CH₂)₆—ATT ATT TTT TAT TTA TTT TTA
TTT TAT TTT ATT TTT TAT T—O—(CH₂)₆—NH—CO—
(CH₂)₂—MB—3′

Prior to sensor fabrication, electrodes (2.0 mm diameter, CH Instruments) were cleaned mechanically by polishing with a 1 μm diamond suspension oil slurry, and then a 0.05 μm alumina oxide powder aqueous solution successively. Each polishing step is followed by sonication of the electrodes in either ethanol or distilled water for 5 min, respectively.

The electrodes were further cleaned electrochemically through successive cathodic and anodic scans in NaOH and H₂SO₄ solutions as follows. First, the electrodes were cleaned in 0.5 μM NaOH by performing repeated cyclic voltammetry scans in the potential window from −1 to −1.6 V (all potentials versus Ag|AgCl) at the scan rate of 1 V s⁻¹ for 300 cycles. Next, the electrodes were moved into a 0.5 M H₂SO₄ solution and performed a 2 V oxidizing chronoamperometric step for 5 s. Subsequently, a reducing potential of −0.35 V for 10 s was applied followed by voltammetric cycles at a scan rate of 4 V s⁻¹ from −0.35 to 1.5 V for 10 scans and then two cycles using the same potential range at 0.1 V s⁻¹.

Finally, the electrodes were washed in deionized water and determined their surface area by acquiring a cyclic voltammogram from −0.35 to 1.5 V at 0.1 Vs⁻¹ in a 0.05 M H₂SO₄ solution.

The E-AB sensors was fabricated using the previously described target-assisted immobilization method (Liu et al., 2021, ACS Appl. Mater. Interfaces, 13:9491-9499). For this, firstly 2 μL of the aptamers as received were reduced to free thiols (100 μM) by adding 4 μL of a 10 mM aqueous solution of tris(2-carboxyethyl) phosphine and stored the tube in the dark at room temperature for 1 h. The reduced aptamers were then diluted in binding buffer (for ATP aptamer: 10 mM HEPES PH 7.6, 100 mM NaCl, and 10 mM CaCl₂); for glucose aptamer: 20 mM HEPES PH 7.6, 1 M NaCl, 5 mM KCl, 10 mM MgCl2, and 5 mM CaCl₂)) to obtain a final concentration of 200 nM. The total aptamer concentration was maintained constant at 200 nM for all experiments to control DNA surface coverage. The aptamer's target was added at a concentration of 0.01 M prior to immersion of the electrochemically cleaned gold electrodes into a 150 UL aliquot of the aptamer-target mixture for overnight incubation in the dark at room temperature.

Following this, the electrodes were rinsed using 1× PBS solution (137 mM NaCl, 2.7 mM KCl, 10 mM Na₂HPO₄, and 1.8 mM KH₂PO₄) to remove any non-specifically adsorbed aptamers. The aptamer-functionalized electrodes were then immersed into the respective binding buffer containing 5 mM 6-mercaptohexanol with the same concentration of corresponding target utilized in the aptamer immobilization step for overnight incubation at room temperature in the dark. Finally, the electrodes were thoroughly rinsed using 1× PBS to remove any non-specifically adsorbed target molecules and stored the electrodes in the binding buffer at room temperature until their use.

Saliva samples were collected following an ethical institutional protocol. Briefly, volunteers collect saliva anonymously following natural drooling in 50 mL falcon tubes. The collected saliva was aliquoted in 5 mL volumes and stored those at −20° C. to avoid freeze-thaw cycles.

Due to E-AB sensor's signal dependence on the interrogating square-wave frequency, responsive frequencies were identified by measuring methylene blue peak current as function of the square-wave frequency. For this, at least three sensors were interrogated in absence and presence of target against frequencies ranging from 2 to 1000 Hz in working buffer and in saliva. The collected peak currents were converted in signal change (%) using the following Eq. 1:

$$Signal Change ( \% ) = \frac{Peak {current}_{bound state} - Peak {current}_{unbound state} \times 100}{Peak {current}_{unbound state}} \qquad ( Equation 1 )$$

Calibration curves were performed in 10 mL of the corresponding working buffer for each aptamer and in 5 mL of undiluted target-spiked saliva. For this square-wave voltammograms were collected from −0.10 to −0.45 V using 25 mV amplitude at either signal-off and signal-on frequencies determined previously.

First, in the absence of target, sensors were interrogated for 40-50 scans until peak currents were stable. Sensors were then challenged to increasing amounts of target (i.e., from 10⁻²⁰ to 10⁻³ M) and record the methylene blue peak current. In the same electrochemical cell, “control” electrodes functionalized with DNA sequences possessing no secondary structures and the same number of nucleotides as the aptamer were also challenged to evaluate for potential target cross-reactivity with methylene blue. Peak currents were extracted at each target concentration using a Python-based script and then performed a non-linear fit of the resulting binding curve to the Hill equation (Eq. 2):

$${C}_{\%}^{[ Target ]} = {C}_{\%}^{0} + ( {C}_{\%}^{Max} - {C}_{\%}^{0} ) \times \frac{[ X ]}{{K}_{D} + [ X ]} \qquad ( Equation 2 )$$

where [X] is the target concentration,

$${C}_{\%}^{[ Target ]} {C}_{\%}^{[ Target ]}$$

is the signal change al a given target concentration,

$${C}_{\%}^{0} {C}_{\%}^{0}$$

is life baseline current observed in the absence of target,

$${C}_{\%}^{Max} {C}_{\%}^{Max}$$

is the signal change at saturating target concentration, and K_{D}K_{D} is the aptamer-target dissociation constant.

It was determined the sensor response time using the signal-on frequency previously determined above and by repeating interrogation every 20 s in target-free saliva. After recording the sensor signal for 200 s, target was injected in saliva and continuously monitored the electrochemical signal for an additional 200 s.

E-AB sensors' response is square-wave frequency dependent. This was observed when interrogating sensors over a broad range of square-wave frequencies (i.e., 2-1000 Hz) in presence or absence of saturating target concentration. Doing so produced two distinct sensor responses where one increases and the other decreases when target is present (FIG. 2). The experiment was repeated in presence of saturating amounts of target (100 μM of adenosine monophosphate and 100 μM of glucose) and calculated the relative signal change of the sensor using Eq. 1 at each of the interrogating frequencies. Doing so resulted in two frequency regions where sensors generated either lower or higher responses to addition of target, which were refer as «Signal OFF» or «Signal ON», respectively. It was observed that deploying sensors in undiluted saliva resulted in larger signal change over the entire range of interrogating frequencies.

When challenging the ATP aptamer sensor with saturating amounts of adenosine monophosphate, the largest signal OFF and signal ON responses were obtained at 12 and 750 Hz, respectively. The glucose sensors, in contrast, produced signal OFF and signal ON responses at 10 and 150 Hz.

E-AB sensor's response is enhanced in undiluted saliva. This was confirmed when repeating glucose and adenosine monophosphate-binding sensor interrogation over a broad range of frequencies (FIG. 2). Doing so also produced a frequency dependent sensor response where the identified responsive frequencies stayed relatively unchanged in comparison to ones measured in buffer. The relative signal change of both sensors increased when deployed in undiluted saliva. The glucose-binding E-AB sensor's signal ON response increased over 4-fold while the adenosine monophosphate binding sensor signal change increased ˜25%.

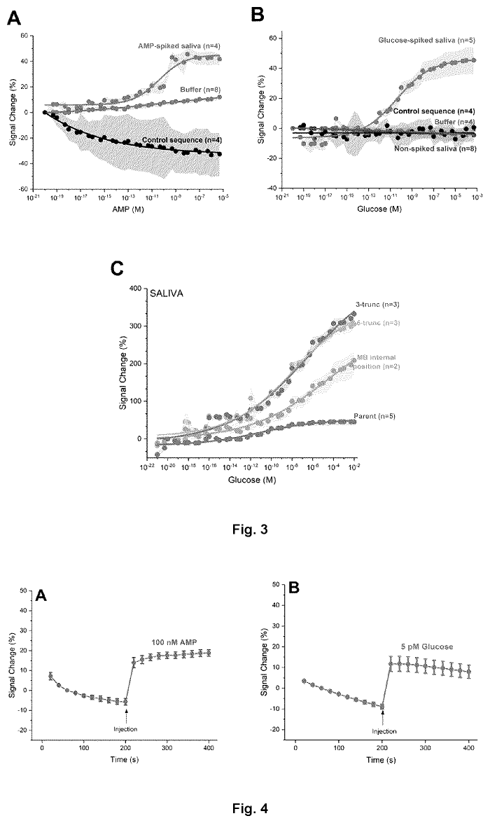

E-AB sensors limits of detection are enhanced when deployed in undiluted saliva. This was confirmed when challenging the adenosine monophosphate and glucose-binding sensors to increasing amounts of the corresponding targets when interrogating at the identified responsive square-wave frequencies above in saliva (FIGS. 3A and 3B). The adenosine monophosphate-binding sensor showed a response with a dynamic picomolar—nanomolar range and a Ko of 0.1 nM while the glucose-binding sensor responded in the femtomolar—nanomolar regime with a Ko of 300 nM. FIG. 3A shows the testing results using the adenosine monophosphate-binding aptamer to increasing amounts of target using a responsive signal ON frequency when deployed in undiluted saliva. Doing so resulted in a maximal signal change of ˜40% with an unprecedented K_{D} of 0.1 nM. When deploying the sensor in buffer, in contrast, no response was measured, highlighting that the ionic condition of saliva offers for unique E-AB sensor performances. Considering that these results show unprecedented limits of detection of E-AB sensors for small molecules, control experiments were performed in which the titration experiment was repeated with electrodes functionalized with a DNA sequence of the same length as the adenosine monophosphate-binding aptamer (i.e., T26) and as the glucose-binding aptamer (i.e., T40) while not having any secondary structures. In both cases, doing so resulted in no relative sigmoidal binding attesting of the specificity of the sensor for adenosine monophosphate and glucose (FIGS. 3A and 3B). Increasing amounts of buffer to the glucose-binding sensor were titrated into saliva to verify that the response was not originating from other buffer constituents (FIG. 3B). E-AB sensors binding glucose also showed enhanced analytical performances when deployed in saliva. When challenging sensors with increasing amounts of glucose a maximal signal change was measured of ˜45% with an unprecedented Ko of 300 nM. When challenging the sensors to increasing amounts of diluted buffer in saliva without glucose present, no signal change was measured, supporting for aptamer specific binding. Again, no change in the sensor response indicative of specific aptamer binding to its target were measured.

To further enhance the glucose sensor analytical performance, the parent aptamer sequence was re-engineered via truncations and modification of the position of methylene blue. Specifically, base pairs were removed at each of the extremities of the aptamer to produce two new variants that are referred herein as 3-trunc and 6-trunc to decrease the distance separating the glucose binding site with the electrode surface. This will allow for enhancement of the glucose aptamer flexibility allowing for an enhancement in methylene blue electron transfer rate and in turn an improved E-AB sensor signal. In addition to aptamer truncations, a glucose-binding variant was also produced in which the original sequence was modified and attached methylene blue internally within the sequence so that it could position closer to the identified aptamer glucose-binding site. When challenging the variants of the glucose aptamer to increasing amounts of target in undiluted saliva, in comparison to the parent variant, all returned higher signal change. While affinities of the variants were larger than the parent variant (K_{D 3-trunc}=300 nM, K_{D 6-trunc}=15 nM, and K_{D MB internal position}=26 μM), the largest increase was observed in response for 3- and 6-trunc variants, which are better candidate for clinically relevant contexts given their dynamic range closer to relevant concentrations.

As demonstrated in FIG. 4, E-AB sensors rapidly respond in the presence of the target.

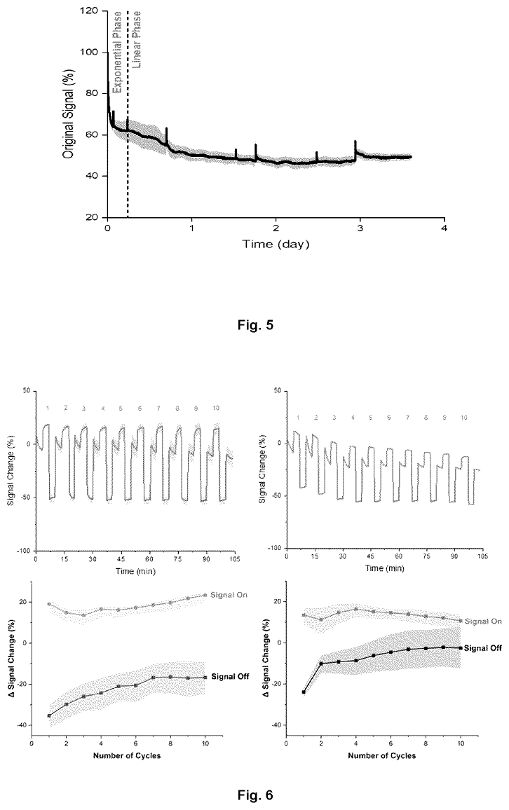

It is further demonstrated as seen in FIG. 5 that E-AB sensor are stable for an extensive period of time (>3 days) when continuously interrogated in undiluted saliva. This was tested by immersing the glucose-binding E-AB sensors in undiluted saliva and recorded square-wave voltammograms at intervals of ˜20 s. In doing so it was observed that the E-AB sensor response initially decreased via an “exponential” phase followed by a “linear” decay prior to signal stabilization. It was hypothesized, as prior literature suggests, that the prior is due to non-specific accumulation of salivary components on the E-AB sensor interface, which lead to ˜40% of the sensor's loss. The linear phase decay, in contrast, was attributed to the electrochemical interrogation of the sensor's interface. Thiols adsorbed on electrodes have a limited stability when interrogated cathodically and this could initiate their desorption. Once that is passed, it was observed that the sensors maintain 50% of their original electrochemical signal for a prolonged period of up to at least 3 days. The E-AB sensors can thus last for longer and withstand the harsh conditions of undiluted saliva. Of note, spikes in the decay correspond to issues associated with the potentiostat which inadvertently disconnected from the biosensors and should be disregarded.

FIG. 6 demonstrate that E-AB biosensors can withstand potentially more than 10 cycles of rinse/target challenge steps directly in undiluted saliva. The adenosine monophosphate-binding (left panels) and glucose-binding (right panels) E-AB sensors were used by first deploying in undiluted saliva. Sensors were then transferred in undiluted saliva containing 1 nM of the respective targets and measured an increase in signal. The sensors were finally rinsed using 1× phosphate buffer prior to repeating these steps for 9 additional times. In doing so it was found that, when using a signal-on interrogating square-wave frequency, the sensors produced a consistent 15-20% response over the 10 cycles. This repetition can last for more rinse/challenge steps and that it demonstrates that E-AB sensors are regenerable. When looking at the signal-on response following each rinsing step (bottom panels), the signal-on square-wave frequency produced consistently 20±5% for the adenosine-monophosphate-binding E-AB sensor and 15±5% for the glucose-binding sensor over these 10 cycles. E-AB sensors can thus be rinsed easily and produce consistent responses for multiple rinse/challenge cycles.

As provided herewith, the E-AB sensors allow measuring glucose levels in saliva. Measuring glucose levels by the provided E-AB sensors allows to monitor diabetes and increased risk of gum disease. Higher levels of glucose encourages bacteria to grow and increases the risk of gums becoming damaged. Similarly there are several other metabolites that are found in saliva that could be used to monitor health status for example carnitines (e.g. 2-Methylbutyryl carnitine, Butyryl carnitine, Isobutyryl carnitine, Propionyl carnitine), lactate/latic acid, malate/malic acid, Adenosine 5′-monophosphate (AMP), maltose etc.

It is further provided that the E-AB sensors as described can be adapted to measure a biomarker or target molecule other than glucose, ATP and/or AMP. As provided, the sensor was adapted to measure vancomycin in saliva. Vancomycin is known to be a glycopeptide antibiotic medication used to treat bacterial infections. Particularly, vancomycin is also taken orally as a treatment for severe Clostridium difficile colitis. Measuring vancomycin level in saliva allows proper dosing and therapeutic monitoring. As provided herewith, a previously reported aptamer binding vancomycin was tested in buffer and its ability to readily deploy in undiluted unstimulated saliva was evaluated. The vancomycin-binding aptamer and its 4-trunc variant is known in the field and has been further adapted in an E-AB sensor (see Dauphin-Ducharme et al., 2019, ACS Sens., 4, 10: 2832-2837). However, no demonstration was made of the applicability of this E-AB sensor in undiluted saliva. Through this, it is provided the analytical figures of merit (sensitivity, dynamic range, limit of quantification, limit of detection) of this biosensor in this complex matrix.

E-AB sensors' response is square-wave frequency dependent. This was observed when interrogating sensors over a broad range of square-wave frequencies (i.e., 2-1000 Hz) in presence or absence of saturating target concentration. Doing so produced two distinct sensor responses where one increases and the other decreases when target is present (FIG. 8). These responsive frequency regimes were identified “signal ON” and “signal OFF”, respectively. When challenging the vancomycin aptamer sensor with saturating amounts of vancomycin, it was observed that the largest signal OFF and signal ON responses were obtained at 10 and 100 Hz, respectively.

E-AB sensor's response is enhanced in undiluted saliva. This was observed when repeating vancomycin-binding sensor interrogation over a broad range of frequencies (FIG. 8). Doing so also produced a frequency dependent sensor response where the identified responsive frequencies stayed relatively unchanged in comparison to ones measured in buffer. However, it was observed that the relative signal change of sensors increased when deployed in undiluted saliva. The E-AB sensor's signal ON response increased over 3-4-fold.

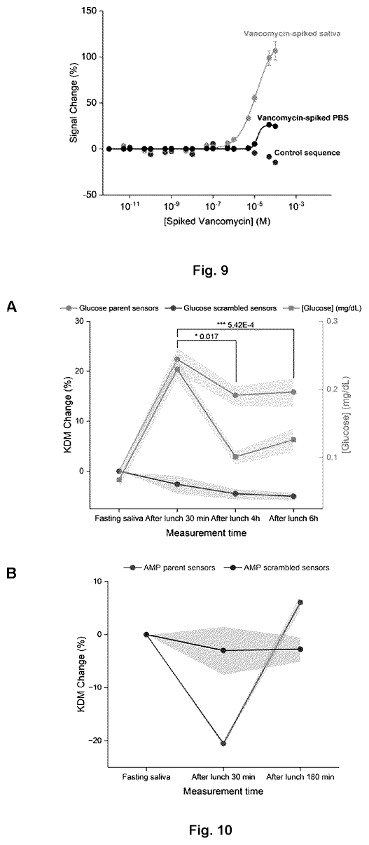

E-AB sensors limits of detection are enhanced when deployed in undiluted saliva. This was observed when challenging the vancomycin-binding sensors to increasing amounts of the corresponding targets when interrogating at the identified responsive square-wave frequencies above in saliva (FIG. 9). The vancomycin E-AB sensor, in contrast, produced a signal gain of ˜25% with a dissociation constant (K_{D}) of ˜17 μM. When in saliva, in contrast to observations in 1×PBS, the vancomycin E-AB sensors generated improved sensor gain of ˜107% and affinities with a K_{D} value of ˜12 μM. Control experiments were performed in which the titration experiment were repeated with electrodes functionalized with a DNA sequence of the same length as the vancomycin-binding aptamer (i.e., T40) while not having any secondary structures. This resulted in no relative sigmoidal binding attesting of the specificity of the sensor for vancomycin (FIG. 9).

As further provided herewith, the variations of glucose and adenosine monophosphate concentrations were monitored as of function of time in saliva samples collected from the same individual at different moments during the same day using E-AB sensors (see FIGS. 10A and B). Doing so, a change in the response of both sensors was observed when saliva samples are collected 30 min after lunch. A decrease and an increase in sensor responses was then measured as time passes after lunch for the glucose and the adenosine monophosphate-binding sensors, respectively, reflecting concentrations of these molecules-high following ingestion and later changes over time. It was validated that the variations of measured signals via E-AB sensors correspond to changes in the endogenous concentrations of glucose using a colorimetric test. Accordingly, the E-AB sensors provided allow to monitor the molecule level variations during a day. The sensors can be regenerated in 7M urea solution followed by DI water rinse and then stored in 1× PBS until next use. Calibration to the signals in fasting saliva could be useful for measuring endogenous molecules.

### Example I

### E-AB Sensors Further Characterization

Since E-AB sensors interrogated in undiluted saliva produced larger signal change than when deployed in buffer, it was hypothesized that this difference in response partly stemmed from differences in the ionic compositions of saliva. The adenosine-monophosphate-binding E-AB sensor were challenged with 1 mM of adenosine-monophosphate while increasing the concentration of divalent cations (Ca²⁺ or Mg²⁺) present in phosphate buffer and recording the resulting sensor response. In doing so, it was determined that the sensor's response changed monotonically with increasing amounts of divalent cations.

Circular dichroism was then used as this technique is sensitive to changes in the chirality of oligonucleotides and has notably been employed to monitor G-quadruplexes and other secondary structures.

The circular dichroism experiment was performed using a J-810 Spectrophotometer (JASCO, MD). For this a quartz cuvette (with a path length of 1 cm) was used in which the aptamer solution was resuspended in 1× phosphate buffer at pH 7.4. The adenosine monophosphate was added at 1 mM and then the divalent cations at the various concentrations using stock solutions containing the same aptamer and buffer concentration with 1 min stirring interval to allow for equilibration. Wavelengths were scanned at 50 nm/min between 230 and 310 nm for every concentrations with a bandwidth of 4 nm and a digital integration time of 4 s, constructing the binding curve using the ellipticity at 265 nm.

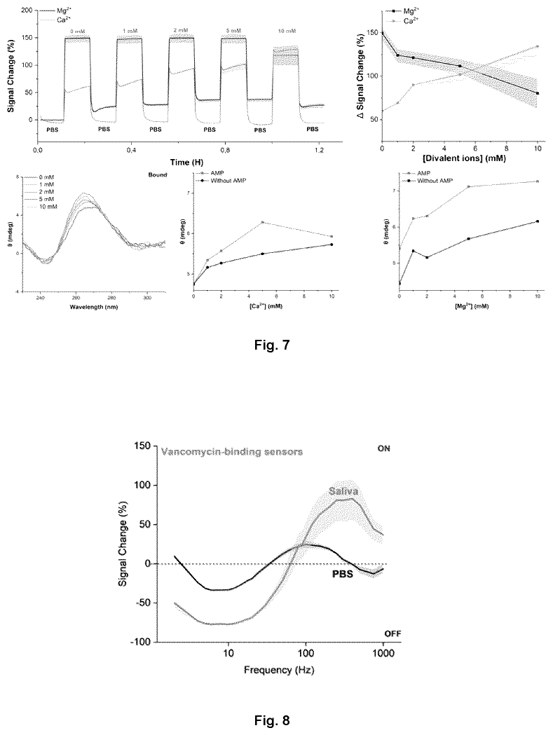

When placing the adenosine-monophosphate-binding aptamer in presence (1 mM) or absence of adenosine monophosphate while increasing the concentrations of divalent cations (Ca²⁺ or Mg²⁺), it was found that the ellipticity measured at 265 nm of a solution of 10 μM of aptamer monotonically increased (see FIG. 7). These results thus further confirmed that the addition of divalent cations (Ca²⁺ or Mg²⁺), typically not included in phosphate buffer solution, plays a critical role in forming the aptamer's tertiary structure that was envision allows for binding to the target. The unique combination of these ions in saliva further enhances the response of E-AB biosensors with respect to buffer as more aptamers form their binding competent structures.

### Example II

### E-AB Sensors for Detecting Vancomycin

Aptamer sequences employed were and purchased from Bio Basic Inc with HPLC purification. The sequences were used without further purification and resuspended at 100 μM in deionized water prior to aliquoting in 2 μL tubes. Bio Basic Inc chemically modified the 5′ end of aptamers with a thiol on a 6-carbon linker and the 3′ end with a carboxy-modified methylene blue attached to the DNA via the formation of an amide bond to a primary amine on a 6-carbon linker.

Vancomycin 4—trunc aptamer:
(SEQ ID NO: 7)
5′ HO—(CH₂)₆—S—S—(CH₂)₆—O—CGA GGG TAC CGC
AAT AGT ACT TAT TGT TCG CCT ATT GTG GGT
CGG—O—(CH₂)₆—NH—CO—(CH₂)₂—MB—3′
T40 control sequence:
(SEQ ID NO: 8)
5′ HO—(CH₂)₆—S—S—(CH₂)₆—O—ATT ATT TTT TAT
TTA TTT TTA TTT TAT TTT ATT TTT TAT—O—
(CH₂)₆—NH—CO—(CH₂)₂—MB—3′

Prior to sensor fabrication, electrodes (2.0 mm diameter, CH Instruments) were cleaned mechanically by polishing with a 1 μm diamond suspension oil slurry, and then a 0.05 μm alumina oxide powder aqueous solution successively. Each polishing step is followed by sonication of the electrodes in either ethanol or distilled water for 5 min, respectively.

The electrodes were then cleaned electrochemically through successive cathodic and anodic scans in NaOH and H₂SO₄ solutions as follows. First, the electrodes were cleaned in 0. μM NaOH by performing repeated cyclic voltammetry scans in the potential window from −1 to −1.6 V (all potentials versus Ag|AgCl) at the scan rate of 1 V s⁻¹ for 300 cycles. Next, the electrodes were moved into a 0.5 M H₂SO₄ solution and performed a 2 V oxidizing chronoamperometric step for 5 s. Subsequently, a reducing potential of −0.35 V for 10 s was applied followed by voltammetric cycles at a scan rate of 4 V s⁻¹ from −0.35 to 1.5 V for 10 scans and then two cycles using the same potential range at 0.1 Vs⁻¹. Finally, the electrodes were washed in deionized water and determined their surface area by acquiring a cyclic voltammogram from −0.35 to 1.5 V at 0.1 V s⁻¹ in a 0.05 M H₂SO₄ solution.

The E-AB sensor were fabricated using the previously described target-assisted immobilization method (see Liu et al., 2021, ACS Appl. Mater. Interfaces, 13, 8: 9491-9499). For this, 2 μL of the as received aptamer was first reduced to free thiols (100 μM) by adding 4 μL of a 10 mM aqueous solution of tris(2-carboxyethyl) phosphine and stored the tube in the dark at room temperature for 1 h. The reduced aptamer was then diluted in binding buffer (10 mM HEPES PH 7.6, 25 mM NaCl, 12.5 mM KCl, 1 mM MgCl2 and 2.5 mM CaCl₂)) to obtain a final concentration of 200 nM. The total aptamer concentration was maintained constant at 200 nM for all experiments to control DNA surface coverage. The aptamer's target was added at a concentration of 0.25 mM prior to immersion of the electrochemically cleaned gold electrodes into a 150 μL aliquot of the aptamer-target mixture for overnight incubation in the dark at room temperature.

Following this, the electrodes were rinsed using 1× PBS solution (137 mM NaCl, 2.7 mM KCl, 10 mM Na₂HPO₄, and 1.8 mM KH₂PO₄) to remove any non-specifically adsorbed aptamers. The aptamer-functionalized electrodes were then immersed into the respective binding buffer containing 5 mM 6-mercaptohexanol with the same concentration of corresponding target utilized in the aptamer immobilization step for overnight incubation at room temperature in the dark. Finally, the electrodes were thoroughly rinsed using 1× PBS to remove any non-specifically adsorbed target molecules and stored the electrodes in the binding buffer at room temperature until their use.

Saliva samples were collected following an ethical institutional protocol. Briefly, volunteers collected saliva anonymously following natural drooling in 50 mL falcon tubes. The collected saliva were aliquoted in 5 mL volumes and stored at −20° C. to avoid freeze-thaw cycles.

Due to E-AB sensor's signal dependence on the interrogating square-wave frequency, responsive frequencies were identified by measuring methylene blue peak current as function of the square-wave frequency. For this, at least three sensors were interrogated in absence and presence of target against frequencies ranging from 2 to 1000 Hz in working buffer and in saliva. The collected peak currents were converted in signal change (%) using Eq. 1.

Calibration curves were performed in 10 mL of the corresponding working buffer for each aptamer and in 5 mL of undiluted target-spiked saliva. For this square-wave voltammograms were collected from −0.10 to −0.45 V using 25 mV amplitude at either signal-off and signal-on frequencies determined previously.

First, in the absence of target, sensors were interrogated for 40-50 scans until peak currents were stable. Sensors were then challenged to increasing amounts of target (i.e., from 10⁻¹¹ to 10⁻⁴ M) and record the methylene blue peak current. In the same electrochemical cell, “control” electrodes functionalized with DNA sequences possessing no secondary structures and the same number of nucleotides as the aptamer to evaluate for potential target cross-reactivity with methylene blue were also challenged. Peak currents were extracted at each target concentration using a Python-based script and then performed a non-linear fit of the resulting binding curve to the Hill equation (Eq. 2).

As seen in FIG. 8, the signal change (relative change in signal upon the addition of saturating target) of E-AB sensors is a strong function of the interrogation square-wave frequency. This was observed when interrogating the vancomycin aptamer in buffer (black curves) and in undiluted human saliva (colored curves) spiked with saturating target concentration over a broad range of frequencies. The relative signal change of the sensor was calculated using Eq. 1 at each of the interrogating frequencies. Doing so resulted in two frequency regions where sensors generated either lower or higher responses to addition of target, which was referred as “signal-off” or “signal-on”, respectively. It was found that deploying sensors in undiluted saliva resulted in larger signal change over the entire range of interrogating frequencies.

### Example III

### E-AB Sensors Characterization

Saliva samples were collected following an ethical institutional protocol (2021-3080). Briefly, volunteers collected saliva anonymously following natural drooling in 50 mL falcon tubes. The fasting saliva sample was collected in the morning after waking up and before toothbrushing or using any oral care products like mouthwash. The other measurements with the saliva were conducted as soon as possible following collection.

The saliva samples were centrifuged at 14000 rpm for 5 min at room temperature. The supernatant was collected and determined using the colorimetric EnzymChrom™ Glucose Assay Kit (EBGL-100), the glucose concentration in saliva as per the described protocol.

Adenosine monophosphate and glucose-binding sensors were interrogated once at a signal-OFF and signal-ON frequencies (10 and 400 Hz) when immersed in the different saliva samples. After each measurement, the sensors were immersed in a 7M urea solution for 10 min followed by copious rinsing with DI water and then stored in 1× PBS until next use. We derived kinetic differential measurement from the peak currents obtained at the two frequencies using Eq. 3.

$$KDM signal = \frac{{Signal}_{ON} - {Signal}_{OFF}}{1 / 2 \times ( {Signal}_{ON} + {Signal}_{OFF} )} \qquad ( Equation 3 )$$

The sensor signal at different time points were normalized to the responses in fasting saliva. In the same electrochemical cell, we fabricated and interrogated electrodes functionalized with DNA sequences having the same number and ratio of nucleotides as the glucose or the adenosine monophosphate-binding aptamer but with the order jumbled (labelled “scrambled sensors” in FIG. 10) to act as negative controls.

While the description has been described in connection with specific embodiments thereof, it will be understood that it is capable of further modifications and this application is intended to cover any variations, uses, or adaptations and including such departures from the present disclosure as come within known or customary practice within the art and as may be applied to the essential features hereinbefore set forth, and as follows in the scope of the appended claims.

1. A sensor for measuring a target molecule level in a fluid sample comprising:
a sensing element comprising a redox reporter attached to at least one aptamer that binds to salivary metabolite; and
a solid surface where the sensing element is attached thereto,
wherein upon binding of the salivary metabolite onto the aptamer, the redox reporter undergoes a binding-induced change in electron transfer.

2. The sensor element of claim 1, wherein the aptamer is attached to the solid surface via a thiol molecule.

3. The sensor element of claim 1, wherein the redox reporter is a methylene blue molecule.

4. The sensor element of claim 1, wherein the salivary metabolite is glucose or vancomycin.

5. (canceled)

6. The sensor element of claim 1, further comprising at least one aptamer binding to a different target molecule.

7. The sensor element of claim 1, wherein the target molecule is adenosine triphosphate (ATP), carnitine, lactate, lactic acid, malate, malic acid, maltose, or adenosine monophosphate (AMP).

8. The sensor element of claim 7, wherein the carnitine is 2-Methylbutyryl carnitine, Butyryl carnitine, Isobutyryl carnitine, or Propionyl carnitine.

9. The sensor element of claim 1, wherein the solid surface is an electrode.

10. The sensor element of claim 1, wherein the electrode is a gold electrode.

11. The sensor element of claim 1, wherein the fluid sample is saliva.

12. The sensor element of claim 1, wherein the sensor element is regenerated for multiple measurements.

13-17. (canceled)

18. A method of measuring at least one metabolite level in a fluid sample from a patient comprising the step of:
contacting the fluid sample with the sensor as defined claim 1; and
monitoring the binding-induced change in electron transfer of the redox reporter indicating the presence of the at least one metabolite in the fluid sample.

19. The method of claim 18, wherein the sample is saliva.

20. The method of claim 18, wherein the electron transfer is monitored via a change in the peak height of square-wave voltammograms.

21. The method of claim 18, wherein the at least one metabolite is glucose, vancomycin, adenosine triphosphate (ATP), carnitine, lactate, lactic acid, malate, malic acid, maltose, or adenosine monophosphate (AMP).

22. The method of claim 21, wherein the carnitine is 2-Methylbutyryl carnitine, Butyryl carnitine, Isobutyryl carnitine, or Propionyl carnitine.

23. The method of claim 18, wherein measuring at least one metabolite level allows monitoring health in the patient.

24. The method of claim 23, wherein measuring glucose level is indicative of the presence or progression of diabetes or increased risk of gum disease in the patient.

25. The method of claim 18, for measuring the at least one metabolite with the sensor for an extended period, or for more than three days.

26. (canceled)

27. The method of claim 25, wherein the metabolite is measured before and/or after the patient consumes food.
